(19) United States
(12) Patent Application Publication
(10) Pub. No.: US 2011/0217728 A1
(43) Pub. Date: Sep. 8, 2011

(54) HIGH THROUGHPUT TEST METHOD FOR EVALUATION OF BIOCIDES AGAINST ANAEROBIC MICROORGANISMS

(76) Inventor: Bei Yin, Buffalo Grove, IL (US)

(21) Appl. No.: 12/674,975

(22) PCT Filed: Sep. 10, 2008
(86) PCT No.: PCT/US08/75755

§ 371 Date: Feb. 24, 2010

Related U.S. Application Data
(60) Provisional application No. 60/973,909, filed on Sep. 20, 2007.

(51) Int. Cl. C12Q 1/18

(52) U.S. Cl. 435/32

(57) ABSTRACT

Provided is a high-throughput method for determining the biocidal efficacy of biocidal agents against anaerobic organisms. The method includes the steps of: providing one or more anaerobe samples in a first set of multiple receptacles accessible to multi-channel pipettes; providing one or more biocidal samples at known concentration(s) in a second set of multiple receptacles accessible to multi-channel pipettes; forming mixtures of the one or more biocidal samples and the anaerobe samples via a multi-channel pipette; incubating the mixtures so as to allow reaction between the biocidal samples and the anaerobe samples; determining each of the one or more biocidal samples' killing (biocidal) effectiveness against the anaerobes at selected time interval(s), wherein each step, except for the step of providing one or more biocidal samples, is conducted under anaerobic conditions.

This application claims priority to U.S. provisional application Ser. No. 60/973,909, filed Sep. 20, 2007, which is incorporated herein by reference in its entirety.

### FIELD OF THE INVENTION

The invention relates to high-throughput test methods for testing the biocidal efficacy of biocidal agents against anaerobic organisms found in common anaerobic environments, such as oil and gas fields.

### BACKGROUND OF THE INVENTION

Anaerobes are organisms that do not require oxygen for growth and may even die in the presence of oxygen. Different levels of aero-tolerance have been reported among anaerobic organisms. Obligate anaerobes are considered to live strictly without oxygen and most of them do not survive more than a few hours during oxygen exposure. Under oxygen stress for even a short time, obligate anaerobic bacteria may experience metabolic changes, morphologic changes, or death.

Sulfate-reducing bacteria (SRB) are classified as obligate anaerobes. SRB use sulfate as the terminal electron acceptor and produce hydrogen sulfide. SRB are commonly found in many environments where anaerobic conditions exist. In industry, SRB can cause many problems in aqueous or water-contacting environments, particularly oil and gas reservoirs, oil and gas wells, oil/gas operation, separation, storage, and transportation systems, oil/gas pipelines, oil/gas vessels, cooling towers, environmental water, wastewater and treatment systems, paper and pulp mill storage & mixing tank and process water, ballast water, other industrial process water, and the like.

As a result of the problems that can be caused by anaerobic organisms, such as anaerobic bacteria, there is a demand for biocidal agents that effectively control anaerobe growth. However, efficacy of biocidal agents is dependent on various factors, including the chemical environment in which the anaerobes are growing and the type or the particular strain of an anaerobe. Thus, a biocidal agent, which may be effective against one strain of a microbial species, may not be similarly effective against a different microbial species or even a different strain of the same species, or under different environmental conditions.

Test methods are therefore widely used for determining the efficacy of biocidal agents against microorganisms from a particular environment. For instance, API RP-38 is the most commonly used industrial standard method recommended by the American Petroleum Institute for SRB evaluation. In API RP-38 testing, a SRB contaminated sample is treated with a test biocidal agent in sealed serum vials. The vials are sealed with septa and aluminum crimp caps to reduce the interference of oxygen in the test method. The samples are then serially diluted for bacterial enumeration. Needle syringes are used for inoculation and transferring aliquots for serial dilution. The incubation time for SRB growth is typically 28 days.

The conventional test methods for biocide evaluation against anaerobes, however, suffer from several disadvantages. For instance, they are time consuming and labor intensive; as noted above, the incubation time for the API test is 28 days. In addition, in part because of the lack of thoroughness in excluding oxygen and the failure to provide favorable atmosphere conditions for anaerobe growth, the known methods do not provide high accuracy and reproducibility. As a result of the complexities and limitations of the conventional methods, a need exists for new, faster, more efficient, and more reproducible testing techniques.

### BRIEF SUMMARY OF THE INVENTION

The invention provides a method for testing the biocidal efficacy of biocidal agents against anaerobes. The method comprises: providing one or more anaerobe samples in a first set of receptacles accessible to multi-channel pipettes; providing one or more biocidal samples at known concentration(s) in a second set of receptacles accessible to multi-channel pipettes; forming mixtures of the one or more biocidal samples and the anaerobe samples via a multi-channel pipette; incubating the mixtures so as to allow reaction between the biocidal samples and the anaerobe samples; determining each of the one or more biocidal samples' killing effectiveness against the anaerobes at selected time interval(s), wherein each step, except for the step of providing one or more biocidal samples, is conducted under anaerobic conditions.

### DETAILED DESCRIPTION OF THE INVENTION

As noted above, the invention provides a high throughput (HTP) test method for measuring the biocidal efficacy of biocidal agents against anaerobic organisms, such as SRB. The method permits testing of the minimal biocidal concentration (MBC) of a particular agent required to achieve complete or certain levels of organism kill after various treatment intervals, under anaerobic conditions, and over time. In some embodiments, the method uses serial dilution techniques, multi-well or multi-tube receptacles accessible to multi-channel pipettes, and a fully functional anaerobic operation system to test the biocidal efficacy.

The HTP test method of the invention provides several advantages compared to conventional methods. For example, because of its high efficiency, the method allows the thorough evaluation and comparison of multiple biocidal agents and agent combinations simultaneously in a short time period, leading to rapid result generation and a reduction in cost of labor and test materials. The capability of testing a large amount of samples also allows additional replicates of each sample and a side-by-side comparison of each agent in a single test, which increases the test accuracy and avoids experimental biases caused by variable test conditions in separate tests.

In addition, the HTP test procedure of the invention provides strict anaerobic growth conditions, which avoids the effect of oxygen on the survival, growth, morphological and physiological status of anaerobes during the entire test period, and leads to quick recovery of anaerobes during enumeration. As a consequence, the HTP method requires much shorter incubation times and further increases test accuracy. For example, only about 3 days incubation time is required for SRB tested in this invention, compared to the 28 days required for the API RP-38 conventional test.

Further, the method of the invention is able to provide efficacy data for the biocide compounds under study at various time intervals, as opposed to the minimum inhibitory concentration (MIC) data of many prior art tests. The method of the invention measures the capability of biocidal agents to kill the microorganisms under testing and over time, whereas an MIC approach only determines the inhibitory effect of the biocidal agent at one fixed time point without providing information on dosages required for lethal effect.

Organisms simply inhibited by biocidal agents can still thrive when the activity of the agents decreases because, for example, of deactivators existing in the environment or the degradation of the agents. Also, organisms under continuous inhibitory treatment can easily develop biocide resistance/tolerance and may survive future treatment with the same or similar types of biocides. Thus data on the dosages of a biocide required to kill microorganism, is particularly important where there is a need to eliminate rather than simply inhibit the organism.

The approach of the invention can also provide more reliable data on biocide efficacy than MIC methods. In a MIC test, the contact of biocidal agents and microorganisms happens in culture media. Some common components in culture media, such as proteins or ammonium compounds, may inactivate certain biocidal compounds, thus artificially interfering with the biocide compound's concentration. In method of the invention, culture medium is required only in the organism enumeration step, where continued activity of residual biocidal agents is not wanted. Therefore, the method of the invention can provide a more reliable evaluation of the actual biocidal agent's efficacy.

The invention provides a method for the high-throughput testing of a biocidal agent's killing efficacy against anaerobe. The method comprises: providing one or more anaerobe samples in a first set of receptacles accessible to multi-channel pipettes; providing one or more biocidal samples at known concentration(s) in a second set of receptacles accessible to multi-channel pipettes; forming mixtures of the one or more biocidal samples and the anaerobe samples via a multi-channel pipette; incubating the mixtures so as to allow reaction between the biocidal samples and the anaerobe samples; determining each of the one or more biocidal samples' killing effectiveness against the anaerobes at selected time interval(s), wherein each step, except for the step of providing one or more biocidal samples, is conducted under anaerobic conditions.

The HTP method of the invention is suitable for testing anaerobic organisms from any environment where such organisms exist including, for example, oil and gas field water, oil and gas field water-based fluids, oil and gas reservoirs, oil and gas operation, separation and transportation systems, oil and gas wells and storage tanks, oil and gas pipelines, oil and gas vessels, fuel, cooling towers, environmental water, soil, wastewater and treatment system, paper and pulp mills, ballast water, industrial equipment, mixing and storage tanks, and process water, paint, latex, coatings, metalworking fluids, aqueous-based slurries and dispersed pigments, adhesives, inks, tape joint compounds, personal care and househood products, other water-containing fluids, biofilms, diagnostic and therapy equipments and tools in medical and healthcare areas, and clinical samples. The anaerobe-containing sample can contain single or mixed species of organisms. Preferred anaerobic organisms, are microorganisms, more preferably bacteria, most preferably SRB. Other suitable anaerobic bacteria include, but are not limited to, anaerobic bacteria commonly found in industrial environments and clinical samples, such as Clostridium, Peptostreptococci, Bacteroides, Actinomyces, Prevotella, Fusobacterium, Leptotrichia, Eubacterium, Bifidobacterium, Lactobacillus, or nitrate and nitrite-reducing bacteria (NRB).

The term “anaerobe samples” in this specification refers to an aqueous sample of anaerobes having a suitable viscosity for testing. The sample may exist naturally in a state that is suitable for testing, i.e., the anaerobes are found existing in a water containing medium having suitable viscosity, or the sample may be prepared artificially by suspending cultured anaerobe(s) in a water-containing medium. The water containing medium for the artificially suspended anaerobe sample may itself be the field environment in which the organism typically resides or contaminates, or it may be prepared artificially. The naturally occurring water containing media are preferably the various environments listed above in which the organisms exist including, but not limited to, oil and gas field water, oil and gas field water-based fluids, oil and gas reservoirs, fuel, cooling tower water, environmental water, wastewater and treatment water, water used in paper and pulp mills, ballast water, industrial process water, paint, latex, coatings, metalworking fluids, aqueous-based slurries and dispersed pigments, water containing adhesives inks and tape joint compounds, personal care and househood products, other water-containing fluids, and clinical samples. When used to prepare the suspension of cultured anaerobes of interest, the water-containing media are preferably sterile before use.

Where the anaerobe naturally exist in a low viscosity liquid environment (and therefore can be transferred using a pipette), further processing of the material is not typically necessary and the anaerobe sample can be directly subjected to the testing of the invention. Direct testing of the anaerobe sample is especially advantageous where the testing can be conducted in close proximity to the sample collection site, or where the transport of the anaerobe sample under anaerobic conditions is possible.

Where there is difficulty in maintaining anaerobic conditions during transportation, it is preferable that the anaerobes be isolated first in a suitable culture medium in a sealed culture bottle and then transported later on. Alternatively the collected anaerobe-containing sample can be added to a suitable anaerobe culture medium in a sealed bottle prior to transport. It is preferred that the culture medium contain a reducing agent. The organisms can then be further enriched in a lab and then be resuspended in deaerated buffer or a medium with suitable viscosity for testing or it can be stored for future evaluation. Anaerobes that exist in non-liquid environments such as a hard-surface or high viscosity materials such as tape joint compounds, can be readily isolated and then resuspended in deaerated buffer or a water containing medium with suitable viscosity for testing or be stored for future evaluation. Alternatively, the anaerobic organisms can be obtained from a commercial source or laboratory collections, and cultured.

The anaerobe sample is distributed into receptacles for biocidal agent testing. This step is conducted in an anaerobic chamber. The anaerobic chamber allows this step and various other steps of the invention to be carried out under an anaerobic environment, which avoids the effect of oxygen on anaerobic organism growth and provides favorable atmosphere conditions for anaerobe growth (atmosphere that is about 90% N₂, 5% CO₂ and 5% H₂) and therefore greatly reduces growth incubation time. Also, because under oxygen stress and toxicity effect anaerobes may experience morphological and physiological changes or death which can lead to change of anaerobe susceptibility to biocides or overestimating the efficacy of biocides, anaerobic conditions as used in the invention leads to more reliable results.

The chamber can be, for example, a glovebox with a work platform or other type of oxygen excluding enclosure that is accessible by hand with or without gloves and which contains a work platform. Advantageously, the chamber should be capable of removing oxygen preferably to a level of less than 2.5% more preferably less than 0.4%. Most preferably, trace amounts of oxygen are removed. Inert gases, such as nitrogen or argon, can be used to provide effective oxygen exclusion.

The chamber preferably contains a built-in incubator, although one is not required. Particularly preferred is an anaerobic chamber that is a unit with a work platform, temperature and condensate controllers, circulated anaerobic gas, trace oxygen removing capability, and a built-in incubator. Such chambers are commercially available. For example, a preferred chamber is the Bactron anaerobic chamber available from Sheldon Manufacturing, Inc. Oregon, USA.

As noted, the anaerobe-containing samples, containing an approximately known amount of anaerobe(s), are distributed into receptacles accessible to multi-channel pipettes inside the anaerobic chamber. Preferred receptacles are multi-well plates. Multiwell plates contain multiple periodic rows and columns of wells for holding individual aliquots of sample. The plates are available in various well numbers and well volumes, such as 6 to 96 wells per plate and 300 ul to 2 mL per well. The samples are placed into the wells of the plates using a pipette, such as a multi-channel pipetting system. Alternatively, an automated or semi-automated device can be placed inside the anaerobic chamber to replace the multi-channel pipettes for use in this and other steps of the method of the invention, including culture medium loading, serial dilutions, inoculation and agent dispersion or transfer.

Optionally, the anaerobe samples may be tested at varying anaerobe concentrations. This may be done by conducting serial dilutions on the anaerobe samples. Such dilutions are preferably done using multi-channel pipettes and multi-well plates as receptacles for the diluted samples and other solutions such as control solutions. In a typical serial dilution procedure, the anaerobe samples, at a particular anaerobe concentration(s), are placed in the top row of wells in a multiwell plate. In the second row of each column, the same anaerobe sample is diluted, e.g., in half. The dilutions are repeated in additional rows of the multiwell plate. Each column of the multiwell plate, therefore, contains the anaerobe(s) at multiple concentrations.

One or more biocidal samples (i.e., biocidal agent present in solution or a matrix) to be tested are prepared directly in or transferred to a second set of receptacles (preferably multiwell plates) accessible to multi-channel pipettes. The biocide samples can be prepared outside the anaerobic chamber and brought inside the anerobic chamber thereafter; however, it is preferred that they be directly prepared inside the anaerobic chamber. Various solvents and matrices can be used for making the samples, including water and organic solvents. In some embodiments, it is advantageous to use the water containing media described above as the matrix for the biocide, since this may provide a good approximation of the conditions under which the biocide will operate. Typically, the medium is sterile or is sterilized before being used to make the biocide sample.

If multiple biocide concentrations are to be tested or MBC is to be measured, multiple serial dilutions of the biocide samples should be carried out thereon. Such dilutions are preferably done using multi-channel pipettes and multi-well plates as receptacles for the diluted samples as well as any other solutions, such as control solutions. In a typical serial dilution procedure, each biocide sample of interest, at a particular concentration, is placed in the top row of wells in a multiwell plate. In the second row of each column, the same biocidal sample is diluted, e.g., in half. The dilutions are repeated in additional rows of the multiwell plate. Each column of the multiwell plate, therefore, contains a particular biocide at multiple concentrations.

The invention is not limited to any particular biocide for testing and any that have the potential for killing the anaerobe of interest are considered particularly suitable. Examples include, but are not limited to, aldehydes such as glutaraldehyde, formaldehyde, phosphonium compounds such as tetrakis hydroxymethyl phosphonium sulphate (THPS), ammonium compounds such as alkyl dimethyl benzyl ammonium chloride, 2,2-dibromo-3-nitrilopropionamide (DBNPA), 2-bromo-2-nitropropane-1,3-diol (Bronopol), thiazolones such as 5-chloro-2-methyl-4-isothiazolin-3-one and 2-methyl-4-isothiazolin-3-one (CMIT/MIT), tris(hydroxmethyl)nitromethane, benzisothiazolinone (BIT), 1-(3-chloroallyl)-3,5,7-triaza-1-azonia-adamantane chloride, 2,6-dimethyl-m-dioxan-4-ol acetate, o-phthalaldehyde, oxazolidines, triazine, and other formaldehyde-releasing biocides, and mixtures of two or more thereof.

Following preparation of the biocide samples, multi-channel pipettes are used to transfer and mix known amounts of the biocide samples with the anaerobe samples. Preferably, this is done by transferring the samples with less volume into the receptacles containing larger volume samples.

Optionally other chemical(s) of interest including, for example, corrosion inhibitor(s), oxygen scavenger(s), scale inhibitor(s), polymer additive(s), can be added into the biocidal samples or the anaerobe samples or the mixtures of these two.

Once contacted, the biocide samples and anaerobe samples are mixed and incubated under anaerobic conditions to allow reaction between the anaerobe and the biocidal agent. Incubation time and/or temperature are selected by the operator to meet the particular needs of interest. Preferably, the incubation temperature is set close to the temperature of the environment from which the tested anaerobe is obtained or where the biocide is planned to be used. The incubation time range depends on the estimated time required for the biocidal agent to start action and the estimated time the activity of the biocidal agent will last under the test conditions. The incubation time can range from seconds to months.

Although not required to be conducted in the anaerobic chamber, the presence of an incubator within the chamber greatly facilitates the incubation step of the invention and helps increase the accuracy of the test as well as shortening the organism enumeration time by avoiding the exposure of the samples to oxygen. During the incubation period, the samples can be reinoculated at different time intervals if desired. Reinoculation can be done, for example, hourly, daily, or weekly depending on the type of field conditions the user is attempting to mimic.

The effectiveness of the biocidal agent(s) can be determined throughout the process (following mixing of the biocide sample with the anaerobe sample), and/or at the end of a selected incubation time period. Effectiveness can be determined either quantitatively, for example, by measuring the reduction of the viable organisms via viable cell enumeration techniques; or qualitatively by inspecting for the complete kill of the organisms. Quantitative and qualitative viable microorganism measuring methods are well known in the art, and the particular method chosen is not critical to the invention.

By way of example, one suitable quantitative measuring technique involves the use of a culture-based serial dilution method. Aliquots of a culture broth suitable for growing the testing anaerobes are placed in arrayed multi-wells or multi-tubes, preferably multiwell plates. To increase the sensitivity or specificity of the test, a growth indicator for a particular organism or an organism group can be added in the culture broth. For example, ammonium ferrous sulfate can be added to culture medium for sulfate reducing bacteria (SRB). When ammonium ferrous sulfate reacts with H₂S produced by SRB during bacterial growth, black precipitates will form, providing a visible indicator of growth and metabolic activity. Also, neutralizing agents which can neutralize the biocidal activity of residual biocidal agent but has no negative effect on the growth of the organism can be added in the culture broth to remove any inhibition effect of the residual biocidal agent on the anaerobes. After different incubation time intervals, or at the end of incubation, part of the anaerobe-biocide sample mixture and any untreated control sample is taken out from each well, using a multi-channel pipette, and transferred into wells of another multiwell plate containing culture broth for further serial dilutions. Alternatively, instead of culture broth, the multiwell plate may contain water or buffer solution. Serial dilutions (one or more) are then made down each column for further cell enumeration.

Following the serial dilutions, the multiwell plate is then incubated to allow any viable bacteria in each well to reach visible growth. Although not required to be conducted in the anaerobic chamber, the presence of an incubator within the chamber greatly facilitates this step of the invention and helps increase the accuracy of the test and shorten the organism enumeration time. Preferably, the incubation temperature is set at the optimum growth temperature, which is typically similar to the temperature of the environment from which the testing anaerobe was collected, or the growth temperature recommended by the institute providing the commercial test strain. The incubation time should not be less than the time required for the untreated control to reach a visible growth, and can be readily ascertained by a person of ordinary skill in the art.

After incubation, the operator records the well number of the last well in each column (counting form the first row to the last row) showing visible growth or growth indication. According to this, the anaerobe population in the original mixture can then be calculated. The viable anaerobe reductions caused by the biocidal effect of each biocide at serial diluted concentration is then calculated by subtracting the anaerobe population in biocidal agent treated sample from that of non-biocide treated control samples. If the residue of the biocidal agent in the culture broth is expected to have impact on the enumeration results due to a residual biocidal activity of the biocidal agent, a neutralization step using neutralizing agents may be applied before or during the enumeration to neutralize the residual biocidal activity.

When water or buffer solution instead of culture broth is used for serial dilutions for anaerobe enumeration, the anaerobe number in each well can be further measured on solid nutrient surface (such as agar plate) using, for example, the Colony Forming Unit test method.

As demonstrated by the Examples below, the enumeration step permits the identification of which biocidal samples being tested exhibit efficacy against anaerobe, as well as the minimal concentration of the agents required to achieve complete or certain levels of anaerobe killing after various treatment intervals.

As an alternative to enumeration, qualitative techniques can be used for determining biocidal efficacy. By way of example, one suitable qualitative measuring technique involving the use of a culture-based method operates analogously to the quantitative method described above except no serial dilution is required. Consequently, the results are read as complete kill instead of amount of reduction of viable anaerobe cells.

Besides the culture-based technique, other techniques that can be used for quantitatively or qualitatively detecting viable organisms, and which are particularly suitable for detection of non-cultivable organisms, include cell ATP (adenosine triphosphate) detection, fluorescent molecule technique, immune assay, nucleic acid detecting technique, and other techniques for detecting cell viability or activity.

Various steps within the process of the invention are conducted under an anaerobic environment. In addition to the anaerobic operation system described above, it is further preferable, particularly when testing obligate anaerobes, that the various solvents, solutions, media, such as buffers, matrices, and water for preparation of anaerobe samples, biocide samples and serial dilutions of these samples, be deaerated prior to use. Deaerated water/buffer/matrix is particularly suitable when anaerobe isolates instead of anaerobe containing field liquid samples are tested. Dearation greatly decreases the effect of oxygen existing in these agents on test results.

Dearation techniques are well known to those skilled in the art and include, for example, inert gas replacement of oxygen, freeze-pump-thaw procedures, oxygen scavenging, and the like. For testing of biocidal efficacy against SRB, it is preferred that the oxygen concentration of deaerated water/buffer/matrix be below 10 ppm, more preferably below 0.5 ppm. For other bacteria, the maximum oxygen level can be higher, depending on the tolerance of the bacteria to oxygen and the optimum atmosphere growth condition the bacteria requires.

Culture media suitable for use in various embodiments of the invention are well known in the art. For instance with SRB, preferable culture media include Starkey's Medium, Sulfate API medium, and Postgate medium. Also with SRB, it is preferred that the concentration of ammonium ferrous sulfate in the SRB inoculum preparation medium be 0.5 to 20 ppm, preferably 1 to 10 ppm. The concentration of ammonium ferrous sulfate in the SRB recovery medium (for enumeration) is preferably 20 to 1000 ppm, more preferably 25 to 100 ppm.

As will be understood by a person of ordinary skill in the art, the sequence of various of the steps of the method of the invention can be varied. For instance, the provision of anaerobe samples into the first set of multiple receptacles can be carried out after, before, or at the same time as, the provision of the one or more biocide samples into the second set of multiple receptacles.

The following examples are provided to further illustrate the invention. The examples are not intended to limit the scope of the invention.

### EXAMPLES

### Example 1

The high throughput test method of the invention is used to evaluate 8 commonly used biocides (Table 1) in oilfield for their effectiveness at controlling field isolated SRB. The biocides are tested in triplicates at 8 different dosage levels. Biocidal efficacy is measured at 9 time intervals over a period of 9 days. Table 2 shows the biocidal efficacy (average of triplicates) of the tested biocides against SRB during the 9-day test period.

TABLE 1
Biocides selected

| Product | Chemical Description |
|---|---|
| UCARCIDE 250 | 50% solution of Glutaraldehyde |
| AQUCAR THPS 75 | 75% solution of Tetrakis hydroxymethyl |
| | phosphonium sulphate |
| UCARSAN 442 | 42.5% Glutaraldehyde and 7.5% alkyl dimethyl |
| | benzyl ammonium chloride solution |
| Barquat MB-80 | 80% solution of alkyl dimethyl benzyl ammonium |
| | chloride (ADBAC) |
| DOW AM 7287 | 20% solution of 2,2-Dibromo-3-nitrilopropion- |
| | amide (DBNPA) |
| BIOBAN BP-30 | 30% solution of 2-Bromo-2-nitropropane-1,3-diol |
| | (Bronopol) |
| CANGUARD CM14 | 14% solution of 5-chloro-2-methyl-4-isothiazolin- |
| | 3-one, 2-methyl-4-isothiazolin-3-one |
| TRIS NITRO 50% | 50% solution of Tris(hydroxmethyl) nitromethane |

Preparation of the Bacterial Suspension and Treatment Plate. the SRB are initially grown anaerobically (37° C.) from a −80° C. frozen stock for 2-3 days in modified Starkey's Medium A broth containing sodium lactate (3.5 g), ammonium chloride (1.0 g), di-potassium, hydrogen orthophosphate (0.5 g), magnesium sulfate (2.0 g), sodium sulfate (0.5 g), calcium chloride (0.1 g), thioglycolic acid (0.1 g), yeast extract (0.5 g), ammonium ferrous sulfate (0.001 g) in 1 L water. The incubation is conducted in the incubator inside a Bactron III anaerobic chamber (Sheldon Manufacturing, Inc. Oregon, USA). A sterile salt solution (1.2490 g NaCl, 2.9290 g NaHCO₃, 0.1910 g Na₂CO₃, 0.0060 g Na₂SO₄, 0.033 g CaCl₂, and 0.0590 g MgCl₂.6H₂O in 1 L water) is prepared, deaerated (repeat the step of vacuuming air out and supplying nitrogen gas) and placed inside the anaerobic chamber at least one day before the test. SRB is collected by centrifuging the culture broth at 2000 g for 15 to 30 minutes. The bacterial pellet is resuspended in the deaerated salt buffer to a final bacterial concentration of ˜10⁷ CFU/mL. The suspension is then added to 96-deep-well plates (Treatment Plates) at 900 μL/well.

Preparation of Biocide Solutions and Biocide Plates. Separate 96-deep-well plates (Biocide Plates) are used to prepare biocide solutions with 8 concentrations as follows: One ml of each biocide solution with the initial concentration (1000 ppm in this case) is added to the first row of the plate in triplicates and 1:2 serial dilutions are made down each column, resulting in 8 gradient levels of biocide concentrations for each biocide.

Treatment of the Bacterial Suspension with Biocide. 100 μL of each biocide solution form the Biocide Plate is transferred to the Treatment Plates and mixed well. The Treatment Plates are covered with sterile 96-well-plate mats and incubated at 37° C. inside the chamber.

Bacterial Enumeration. The viable bacteria in each well are enumerated at 9 time intervals over a period of 9 days, using the serial dilution method. At each time interval starting from 2 hours, 20 μL aliquots are sampled from each well of the Treatment Plates and transferred into the first row of 96-well microtiter plates filled with modified Starkey's Medium A broth 2 (180 μL per well) containing sodium lactate (3.5 g), ammonium chloride (1.0 g), di-potassium, hydrogen orthophosphate (0.5 g), magnesium sulfate (2.0 g), sodium sulfate (0.5 g), calcium chloride (0.1 g), thioglycolic acid (0.1 g), ammonium ferrous sulfate (0.025 g) in 1 L water. 1:10 serial dilutions are then made down each column and the plates are incubated at 37° C. inside the chamber. The Treatment Plates are reinoculated with SRB suspension at the final SRB level of ˜10⁷ CFU/mL at day seven after bacterial enumeration on that day. After three days of incubation, the well number of the last well in each column showing growth and black precipitate (indicating SRB growth) is recorded. The bacterial log reduction caused by the biocidal effect of each biocide is calculated by subtracting the last growth well number recorded for the biocide treated sample from that of the non-biocide treated control sample.

The entire test of this example can be conducted inside the anaerobic chamber. Table 2 shows the biocidal efficacy of the 8 biocides against the SRB field cultures during 9 days of test period.

TABLE 2
Biocide concentration (ppm, active) required to achieve at least 3 log
reduction of SRB during 9 days of test period

| | Time intervals | | | | | | | | |
|---|---|---|---|---|---|---|---|---|---|
| Biocide | 2 hrs | 4 hrs | 24 hrs | 48 hrs | 72 hrs | 96 hrs | 168 hrs | 192 hrs | 216 hrs |
| UCARCIDE | 12.5 | 12.5 | 12.5 | 12.5 | 25 | 25 | 25 | 25 | 50 |
| 250 | | | | | | | | | |
| AQUCAR | 50 | 25 | 25 | 12.5 | 6.3 | 6.3 | 6.3 | 12.5 | 25 |
| THPS 75 | | | | | | | | | |
| UCARSAN | 25 | 25 | 12.5 | 12.5 | 12.5 | 12.5 | 12.5 | 12.5 | 25 |
| 442 | | | | | | | | | |
| BARQUAT | 6.3 | 6.3 | 6.3 | 6.3 | 6.3 | 6.3 | 6.3 | 6.3 | 12.5 |
| CB-80 | | | | | | | | | |
| DOW AM | 6.3 | 6.3 | 12.5 | 12.5 | 12.5 | 12.5 | 12.5 | 25.0 | 50 |
| 7287 | | | | | | | | | |
| BIOBAN BP- | 50.0 | 50.0 | 50.0 | 50.0 | 50.0 | 50.0 | 50.0 | 100.0 | >100.0 |
| 30 | | | | | | | | | |
| CANGUARD | 50.0 | 50.0 | 50.0 | 50.0 | 50.0 | 50.0 | 50.0 | 50.0 | 100.0 |
| CM 14 | | | | | | | | | |
| TRIS NITRO | 100.0 | 50.0 | 50.0 | 50.0 | 50.0 | 50.0 | 25.0 | 25.0 | 50.0 |
| 50% | | | | | | | | | |

The Example 1 study demonstrates some of the advantages of the HTP test method of the invention in evaluating and comparing multiple biocides and their blends. In total, Example 1 generates 1728 data points, which includes the log reduction of SRB treated by 8 biocides in triplicates at 8 dosage levels for 9 time intervals. If the same test is conducted using the conventional API RP-38 test method, about 78 days including about 280 working hours of a well trained technician are required to obtain the final results. In contrast, just about 16 days including about 12 working hours of a well trained technician is required using the HTP test method of the invention as practiced in Example 1.

### Example 2

Glutaraldehyde (UCARCIDE 250) is repeatedly tested in two separate tests for biocidal efficacy against SRB in oilfield produced water using a conventional serum vial dilution test method for comparative purposes. Water samples are treated with 12.5 ppm and 25.0 ppm of Glutaraldehyde for three days in GasPak anaerobic jar. Following the treatment, viable SRB bacteria are enumerated by serial dilution method. 1 mL mixture is transferred using needle syringe into 9 mL of SRB culture medium in SRB culture vials sealed with septa and aluminum crimp caps. The culture vials are then incubated for 14 days. SRB log reduction is recorded in Table 3.

TABLE 3
Efficacy of glutaraldehyde (UCARCIDE 250) tested in two separated
tests using conventional glass vial dilution method

| | Log reduction of SRB 3 days after biocide addition | | | |
|---|---|---|---|---|
| Glutaraldehyde | Water sample 1 | | Water sample 2 | |
| concentration | 1^{st} test | 2^{nd} test | 1^{st} test | 2^{nd} test |
| (ppm, active) | result | result | result | result |
| 12.5 | 5 | 4 | 1 | 5 |
| 25.0 | 1 | 5 | 2 | 5 |

### Example 3

In another study, Glutaraldehyde (UCARCIDE 250) is repeatedly tested in two separate tests for biocidal efficacy against a SRB culture, using the high throughput test method of the invention. The same test procedure described in Example 1 is used. Log reduction of SRB after a 3 hour treatment by glutaraldehyde at five gradient dosage levels is presented in Table 4. As can be seen, the HTP method of the invention generates data with higher reproducibility compared to the conventional test method.

TABLE 4
Efficacy of glutaraldehyde (UCARCIDE 250) tested
in two separated tests using the HTP method

| | |
|---|---|
| Glutaraldehyde | Log reduction of SRB 3 |
| concentration | hours after biocide addition |

| (ppm, active) | 1st test | 2nd test |
|---|---|---|
| 100.0 | 5.6 | 6.3 |
| 66.7 | 4.9 | 5.3 |
| 44.5 | 3.9 | 4.3 |
| 29.7 | 3.2 | 4.0 |
| 19.8 | 0.9 | 0.3 |

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A method for testing the biocidal efficacy of biocidal agents against anaerobes, the method comprising:
providing one or more anaerobe samples in a first set of multiple receptacles accessible to multi-channel pipettes;
providing one or more biocidal samples at known concentration(s) in a second set of multiple receptacles accessible to multi-channel pipettes;
forming mixtures of the one or more biocidal samples and the anaerobe samples via a multi-channel pipette;
incubating the mixtures so as to allow reaction between the biocidal samples and the anaerobe samples;
determining each of the one or more biocidal samples' killing effectiveness against the anaerobes at selected time interval(s),
wherein each step, except for the step of providing one or more biocidal samples, is conducted under anaerobic conditions.

2. The method of claim 1 wherein the step of providing the one or more biocide samples in a second set of multiple receptacles is conducted under anaerobic conditions.

3. The method of claim 1 wherein the step of determining each of the one or more biocidal samples' effectiveness is conducted at various time intervals throughout the incubation step or is conducted at the end of incubation step.

4. The method of claim 1 wherein the step of determining each of the one or more biocide samples' killing effectiveness is conducted by either quantitatively measuring viable anaerobe or by qualitatively measuring whether anaerobe kill is complete.

5. The method of claim 1 wherein the mixtures of one or more biocidal samples and anaerobe samples are re-inoculated with anaerobe sample(s) during the incubation step.

6. The method of claim 1 wherein the one or more biocidal samples comprise biocide agents in water containing medium naturally occurring or artificially prepared.

7. The method of claim 1 wherein the one or more biocide samples are provided at varying concentrations.

8. The method of claim 7 wherein the one or more biocide samples are prepared directly in a set of multiple receptacles via serial dilution techniques.

9. The method of claim 1 wherein the source of anaerobes is oil and gas field water, oil and gas field water-based fluids, oil and gas reservoirs, oil and gas wells, oil and gas operation, separation, storage, and transportation systems, oil and gas pipelines, oil and gas vessels, fuel, cooling towers, environmental water, soil, wastewater and treatment system, paper and pulp mills, ballast water, industrial equipments, mixing and storage tanks, and process water, paint, latex, coatings, metalworking fluids, aqueous-based slurries and dispersed pigments, adhesives, inks, tape joint compounds, biofilms, personal care and household products, other water-containing fluids, hard surface, and diagnostic and therapy equipments and tools in medical and healthcare areas, or clinical samples.

10. The method of claim 1 wherein the source of water containing media used for preparing anaerobe or biocide samples is oil and gas field water, oil and gas field water-based fluids, oil and gas reservoirs, fuel, cooling tower water, environmental water, wastewater and treatment water, water used in paper and pulp mills, ballast water, industrial process water, paint, latex, coatings, metalworking fluids, aqueous-based slurries and dispersed pigments, water containing adhesives inks and tape joint compounds, personal care and househood products, other water-containing fluids, or clinical samples.

11. The method of claim 1 wherein the anaerobe in the anaerobe samples is a single species or mixture species of anaerobic organisms.

12. The method of claim 1 wherein the anaerobe in the anaerobe samples is a single species or mixture species of Clostridium, Peptostreptococci, Bacteroides, Actinomyces, Prevotella, Fusobacterium, Lactobacillus, Leptotrichia, Eubacterium, Bifidobacterium, species from sulfate reducing bacteria, or species from nitrate and/or nitrite-reducing bacteria.

13. The method of claim 1 wherein the one or more anaerobe samples are naturally occurring anaerobe containing liquid materials or are prepared by mixing anaerobe containing material or anaerobe culture(s) with water containing medium naturally occurring or artificially prepared.

14. The method of claim 1 wherein the one or more anaerobe samples are provided at varying anaerobe concentrations.

15. The method of claim 1 wherein the biocidal agent is one or more of aldehydes such as glutaraldehyde, formaldehyde, phosphonium compounds such as tetrakis hydroxymethyl phosphonium sulphate (THPS), ammonium compounds such as alkyl dimethyl benzyl ammonium chloride, 2,2-dibromo-3-nitrilopropionamide (DBNPA), 2-bromo-2-nitropropane-1,3-diol (Bronopol), thiazolones such as 5-chloro-2-methyl-4-isothiazolin-3-one and 2-methyl-4-isothiazolin-3-one (CMIT/MIT), tris(hydroxmethyl)nitromethane, benzisothiazolinone (BIT), 1-(3-chloroallyl)-3,5,7-triaza-1-azonia-adamantane chloride, 2,6-dimethyl-m-dioxan-4-ol acetate, o-phthalaldehyde, oxazolidines, triazine, and other formaldehyde-releasing biocides, and mixtures of two or more thereof,

16. The method of claim 1 wherein the first set of multiple receptacles and the second set of multiple receptacles are independently multi-well receptacles or multi-tubes.

17. The method of claim 1 wherein the first set of multiple receptacles and the second set of multiple receptacles are multiwell plates.
